(19) United States
(12) Patent Application Publication
(10) Pub. No.: US 2010/0179184 A1
(43) Pub. Date: Jul. 15, 2010

(54) ARTICLE OF MANUFACTURE FOR PRASUGREL

(76) Inventors: Matthew John Moon, Indianapolis, IN (US); Peter Lloyd Oren, Fishers, IN (US)

Correspondence Address: ELI LILLY & COMPANY, PATENT DIVISION, P.O. BOX 6288, INDIANAPOLIS, IN, 46206-6288, US

(21) Appl. No.: 12/445,876

(22) PCT Filed: Dec. 4, 2007
(86) PCT No.: PCT/US07/86358

§ 371 Date: Apr. 16, 2009

Related U.S. Application Data
(60) Provisional application No. 60/868,993, filed on Dec. 7, 2006.

(51) Int. Cl. A61K 31/4365; A61P 7/02; B65D 85/00

(52) U.S. Cl. 514/301; 206/524.2

(57) ABSTRACT

An article of manufacture comprising packaging prasugrel tablets, caplets, capsule or other solid form of prasugrel in an air and/or moisture impervious container under a positive liquid gas pressure.

### FIELD OF THE INVENTION

The invention relates to an article of manufacture of prasugrel, a thienopyridine platelet aggregation inhibitor.

### BACKGROUND OF THE INVENTION

Thienopyridines such as ticlopidine and clopidogrel (sold as Plavix®, or Iscover® registered trademarks of Sanofi-Aventis S.A.) have been used for the treatment of thrombosis.

Prasugrel is a next generation thienopyridine currently undergoing clinical development for the treatment or prevention of thrombosis and/or related diseases including as an adjunct to percutaneous coronary intervention procedures.

U.S. Pat. No. 6,688,468 B2 discloses the use of oxygen scavengers to enhance the stability and shelf life of drugs.

Prolonged exposure of prasugrel to air and/or moisture results in some degradation associated with stability further resulting in shorter shelf life. In the past, containers including bottles have been packaged with tablets, caplets or capsule under inert atmospheres. Bottles can be inerted using conventional gases. However, it is often difficult to adapt conventional gas inerting of bottles to standard high speed bottle packaging lines and obtain the desired minimal oxygen concentrations in the bottles. Therefore, there is a need for a process for packaging prasugrel and other air and/or moisture sensitive pharmaceutical agents to achieve the desired minimal oxygen concentrations in the containers, and operable in standard high speed bottle packaging lines. Achieving the desired minimal oxygen concentration in the headspace of the container would translate to improvements in the stability and shelf life of prasugrel and other air and/or moisture sensitive pharmaceutical agents.

### SUMMARY OF THE INVENTION

The present invention relates to an article of manufacture comprising tablets, caplets, capsules or other solid form of prasugrel or an air and/or moisture sensitive pharmaceutical agent packaged in an air and/or moisture impervious container under a positive liquid gas pressure.

The present invention provides an article of manufacture of prasugrel

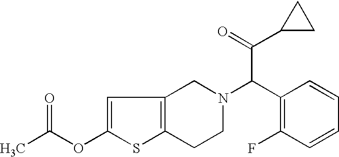

comprising tablets, caplets, capsules or other solid form of prasugrel packaged in an air and/or moisture impervious container under a positive liquid gas pressure.

The present invention relates to a process for improving the stability of a tablet, caplet, capsule or other solid form of prasugrel comprising the steps of:

a. placing a tablet, caplet, capsule or other solid form of prasugrel in an air and/or moisture impervious bottle;
b. optionally adding a desiccant;
c. optionally adding an oxygen scavenger,
d. adding liquid nitrogen; and
e. sealing the air and/or moisture impervious bottle under a positive liquid nitrogen pressure.

The present invention relates to a process for improving the shelf life of tablets, caplets, capsules or other solid form of prasugrel comprising the steps of:

a. placing a tablet, caplet, capsule or other solid form of prasugrel in an air and/or moisture impervious container;
b. optionally adding a desiccant;
c. optionally adding an oxygen scavenger,
d. adding liquid nitrogen; and
e. sealing the air and/or moisture impervious container under a positive liquid nitrogen pressure.

The present invention provides a process for the manufacture of tablets, caplets, capsules or other solid form of prasugrel containing from about 5 mg to about 10 mg of prasugrel comprising the steps of:

a. placing tablets, caplets, capsules or other solid form of prasugrel in an air and/or moisture impervious bottle;
b. optionally adding a desiccant;
c. optionally adding an oxygen scavenger;
d. adding liquid nitrogen, and
e. sealing the air and/or moisture impervious bottle under a positive liquid nitrogen pressure.

The present invention relates to a process for manufacturing prasugrel comprising packaging prasugrel tablets, caplets, capsules or other solid form of prasugrel each containing from about 5 mg to about 10 mg base equivalent per tablet, caplet, capsule, or other solid form of prasugrel, in an air and/or moisture impervious container under positive liquid nitrogen pressure comprising the steps of:

a. placing said tablets, caplets, capsules or other solid form of prasugrel in an air and/or moisture impervious container;
b. adding liquid nitrogen,
c. optionally adding a desiccant,
d. optionally adding an oxygen scavenger; and
e. sealing the bottle under a positive liquid nitrogen pressure.

The present invention relates to a process for the manufacture of prasugrel comprising the steps of:

a. placing tablets, caplets, capsules or other solid form of prasugrel in an air and/or moisture impervious container;
b. adding liquid nitrogen; and
c. sealing the air and/or moisture impervious container under a positive liquid nitrogen pressure.

The present invention relates to an article of manufacture of prasugrel comprising packaging prasugrel tablets, caplets or capsules each containing from about 5 mg to about 60 mg base equivalent per tablet, caplet, capsule, or other solid form of prasugrel, in an air and/or moisture impervious container under positive liquid nitrogen pressure.

The present invention relates to an article of manufacture of prasugrel comprising packaging prasugrel tablets, caplets, capsules or other solid form of prasugrel each containing from about 5 mg to about 10 mg base equivalent per tablet, caplet, capsule, or other solid form of prasugrel, in an air and/or moisture impervious container under positive liquid nitrogen pressure wherein the container is a bottle, and/or wherein the oxygen content of the headspace in the bottle is less than 5%, and preferably less than 4%.

The present invention relates to a pharmaceutical kit comprising tablets, caplets, capsules or other solid form of prasugrel packaged in an air and/or moisture impervious container under a positive liquid nitrogen pressure.

As used herein the term “prasugrel” means the compound of formula I as the hydrochloric acid salt, the base or a mixture thereof.

The term “base equivalent” as used herein is an amount of prasugrel that, if packaged as the HCl salt, is the mole equivalent amount of the base form per tablet, caplet, capsule, or other solid form of prasugrel. One of skill in the art is able to make the conversion and sample equivalent amounts are shown in the examples.

The term “other solid form” as used herein includes fast disintegrating, fast dissolving, quick release or other approved or approvable solid presentations (including semi-solid presentations such as lyophilized formulations) of prasugrel. Methods of preparing said solid formulations are known to one of skill in the art.

The phrase “distance from nozzle to cap” refers to the distance along a moving processing line from the bottom of the liquid nitrogen dosing nozzle to the top of the bottle opening. If this distance is too far, the liquid nitrogen may evaporate before entering the container to displacing the oxygen or may displace the oxygen less efficiently. One of skill in the art is able to adjust the distance from the nozzle to cap to achieve improved efficiency in the process of adding liquid nitrogen to the container and displacing the oxygen.

The phrase “distance from nozzle to capping” refers to the distance from the liquid nitrogen dosing nozzle to the point at which the cap is applied to the containers and then induction sealed. If this distance is too long, the bottles may begin to regain the oxygen that was displaced initially. If the distance is too short, then the containers (especially plastic bottles) may be overly pressurized and have a puffy appearance. One of skill in the art is able to adjust the distance from the nozzle to capping and/or the liquid nitrogen dosing rate to achieve improved efficiency in the filling and capping process and avoid overly pressurized bottles.

The phrase “air and/or moisture impervious container” as used herein, means a container which is resistant to the permeation of air and/or moisture. Examples of air and/or moisture impervious containers include bottles, preferably, multilayer bottles containing HDPE (high density polyethylene) or polypropylene in conjunction with a layer resistant to oxygen permeation such as EVOH (ethylene-vinyl alcohol copolymer). Other suitable multi-layer bottles could be prepared using polymers such as of “COC” or “COP” (cyclic olefin copolymers—COC, cyclic olefin polymer—COP) with a layer resistant to oxygen permeation such as nylon. These multi-layer bottles may consist of two or more layers and may have additional additives to promote the adhesion and structural integrity of the bottle itself Bottles having high resistance to air and/or moisture are often referred to as barrier bottles and are advertised and sold as such.

Glass and aluminum containers that are resistant to air and/or moisture permeation are also within the meaning of “air and/or moisture impervious containers.” The primary requirement is that the containers, preferably bottles are capable of being adequately sealed with an induction foil seal or other suitable means so that the resulting packages have the ability to prevent air and/or moisture permeation. One of skill in the art is aware that absolute imperviousness to air and/or moisture may be difficult and/or impractical to achieve. The phrase “air and/or moisture impervious container” is used comparatively based on the knowledge of one skilled in the art that some materials are less impervious to air and/or moisture than others and that absolute imperviousness is difficult to attain. A most preferred container is a bottle. A most preferred bottle is a barrier bottle. A preferred barrier bottle is glass or a multilaminate bottle. A multilaminate bottle consisting of HDPE/EVOH/HDPE optionally with additional layers is most preferred based on the low degree of moisture and oxygen permeability and because these bottles are not as fragile as glass and unlike aluminum bottles can be readily induction sealed.

The phrase “positive liquid gas pressure” as used herein means the use of liquefied gases including liquid nitrogen, liquid argon, liquid helium, and liquid carbon dioxide. These liquefied gases expand when added to a container and reduce the oxygen level in the container by displacing the air in the container. A most preferred liquid gas for the practice of the invention is liquid nitrogen.

The phrase “positive liquid nitrogen pressure” as used herein means that the volume of space surrounding the tablet, caplet, capsule or other solid form of prasugrel in the bottle is essentially, nearly, or as much as practically possible completely filled with gaseous liquid nitrogen (gaseous nitrogen derived from liquid nitrogen by evaporation) and capped in time to maintain the displacement effect (positive pressure) of liquid nitrogen expansion. Thus, the term “under a positive liquid nitrogen pressure” means that the packaging of the bottle including but not limited to sealing and capping is performed wholly or partially under an atmosphere of gaseous nitrogen derived from expansion of the added liquid nitrogen. The application of liquid nitrogen during the packaging process provides the positive liquid nitrogen pressure necessary for the practice of the invention. Preferably, the barrier bottle is (1) packed with prasugrel, (2) optionally packed with a desiccant, (3) dosed with an appropriate quantity of liquid nitrogen, and (4) sealed and/or capped under an atmosphere (blanket) of gaseous liquid nitrogen (in that order) such that the headspace of the bottle contains a significant preponderance of gaseous nitrogen derived from liquid nitrogen. More preferably, the barrier bottle is (1) packed with prasugrel, (2) optionally packed with a desiccant, (3) optionally packed with oxygen scavenger (4) dosed with an appropriate quantity of liquid nitrogen, and (5) sealed and/or capped under an atmosphere (blanket) of gaseous liquid nitrogen (in that order) such that the headspace of the bottle contains a significant preponderance of gaseous nitrogen derived from liquid nitrogen. The liquid nitrogen is necessary to effect a more complete displacement of air and/or moisture upon expansion (approximately 700 fold) during and after the filling and sealing/capping process.

More preferably, the barrier bottle may be purged with liquid nitrogen and the solid dosage form of prasugrel is added under the positive liquid nitrogen pressure (blanket of gaseous nitrogen derived from liquid nitrogen) followed by optional addition of desiccant, optional addition of oxygen scavenger, and followed by sealing and capping under a positive pressure of gaseous nitrogen derived from liquid nitrogen. It is also preferable that the packaging including filling, sealing, and capping be completed within a reasonable time limit such that the displacement of oxygen by liquid nitrogen is maintained. A reasonable time for the packaging is from a few seconds to a few minutes but more preferably from a few seconds to a less than 15 seconds after dosing with liquid nitrogen and is usually dependent upon the bottle contents and the ability of the contents to impact the volatilization of the liquid nitrogen dose and the amount and/or extent of liquid nitrogen dosing. After packaging (including sealing) the bottle may have a slight bulge (depending on the rigidity of the particular bottle) due to the expansion of the liquid nitrogen upon warming (temperature equilibration). Such bulging bottles caused by liquid nitrogen expansion are within the scope of the invention. It may be necessary to control the amount of nitrogen pressure such that the gas-filled bottle does not bulge or is not pressurized to the extent of affecting further packaging, palleting or storage operations. The positive liquid nitrogen pressure helps to ensure a pressure gradient disfavoring and minimizing entry of air and/or moisture into the bottle, thus manifesting the benefit of the invention.

In the past, bottles have been packaged with tablets, caplets or capsule under inert atmospheres. While bottles can be inerted using non-liquefied gases, it is often difficult to adapt this process to standard high speed bottle packaging lines to obtain the desired minimal oxygen concentrations in the bottles. Table 1, for example, shows some packaging trials conducted on a conventional filling line that is fitted with a nitrogen tunnel (consisting of a stainless steel enclosure to cover the filling belt with the bottles and containing perforations for the delivery of gaseous nitrogen) in addition to a liquid nitrogen dosing device.

TABLE 1
Gaseous Nitrogen Tunnel in Conjunction with Liquid Nitrogen Dosing
VBS Parameters

| Bottle Info | | | Liquid Nitrogen Delivery | | | | | % Oxygen |
|---|---|---|---|---|---|---|---|---|
| | Tablet | Tri- | Device | | Line | Time Delay | Nitrogen Tunnel and | Content Mean |
| Contents | Count | Sorb | Nozzle | Dosing Duration | Speed | Nozzle to Capper | Cap Purge (on/off) | Value |
| Placebo | 4 | 2 g | 0.105″ | 60 millisec | 32 ft/min | 8.8 sec | off | 2.40 |
| Placebo | 4 | 2 g | 0.105″ | 60 millisec | 32 ft/min | 8.8 sec | on - ~20-25 psi | 8.59 |

The operation of this nitrogen tunnel in conjunction with liquid nitrogen dosing leads to higher oxygen values in the bottles (after subsequent capping and induction sealing) than using liquid nitrogen alone. These results are consistent with earlier trials with a gaseous (ambient) nitrogen tunnel alone that demonstrate difficulty in achieving less than 10% oxygen concentrations in the sealed bottles.

Based on these investigations, applicants conclude that attempting to displace oxygen from the bottle headspace with ambient (regular) gaseous nitrogen or other ambient inert gases would require slower production speeds to be employed, result in less than adequate displacement of oxygen in the bottle headspace, and in addition may require an indexing mechanism to introduce the inert gas into the lower part of the bottle to then displace the oxygen contained in the bottle. Furthermore, the heat required and/or generated during capping and induction sealing operations potentially may result in displacement of ambient inert gases (such as ambient nitrogen gas) from the bottle than with the cooler liquid nitrogen. Thus, the invention employs the advantages of cooler and more expandable liquid gases, particularly, liquid nitrogen compared to ambient inert gases. Applicants have discovered that the use of liquid nitrogen is a more effective method of displacing air and/or moisture thereby improving the stability and shelf life of prasugrel. The use of liquid nitrogen is a preferred and advantageous means for displacing the air contained in normal bottle headspace because liquid nitrogen expands to approximately 700 times its volume upon introduction into a bottle and warming to ambient conditions. The expansion of liquid nitrogen can be very effective in reducing the oxygen concentration in packaging configurations.

One embodiment of the present invention is a pharmaceutical formulation comprising prasugrel wherein tablets, caplets or capsules of prasugrel are packaged in a barrier bottle under a positive liquid nitrogen pressure or atmosphere. A particularly preferred embodiment of the invention is the packaging of tablets, caplets or capsules of prasugrel under positive nitrogen pressure in multilayer bottles such as for example a bottle made of or coated with one or more layers of HDPE, EVOH, COC, COP or nylon.

In another preferred embodiment, the tablets, caplets or capsules of prasugrel are packaged in a barrier bottle containing a desiccant and sealed/capped under positive liquid nitrogen.

In yet another preferred embodiment, the tablets, caplets or capsules of prasugrel are packaged in a barrier bottle containing a desiccant and an oxygen scavenger wherein the bottle is sealed/capped under positive liquid nitrogen pressures.

A solid oral dosage form of prasugrel may be prepared using a variety of pharmaceutically acceptable excipients known to one skilled in the art. Preferably, one or more excipients would be selected from each of the following categories:

a. Diluents such as but not limited to mannitol, lactose monohydrate, pregelatinized starch or microcrystalline cellulose.
b. Disintegrants such as but not limited to croscarmellose sodium, low substituted hydroxypropyl cellulose or sodium starch glycolate.
c. Binders including but not limited to hydroxypropyl methylcellulose and hydroxypropyl cellulose. For tablet applications, a lubricant would also be recommended such as but not limited to magnesium stearate, stearic acid, and glyceryl behenate.

If a tablet is produced, it is often desirable to film-coat the resulting tablet to provide a pharmaceutically acceptable appearance and to make said tablet easier to swallow. Commercial suppliers such as, for example, Colorcon Inc. (USA), produce a variety of film coating systems containing polymers, plasticizers and pigments that can be mixed with water and sprayed onto the tablets in a side vented coating pan. A particularly preferred system is marketed as Opadry (Colorcon Inc). The Colorcon film coating system (containing the additive lactose monohydrate) is especially useful in film coating debossed tablets.

Prasugrel may be blended with one or more excipients described above and filled into capsules or compressed into tablets or caplets. One of skill in the art is able to manufacture prasugrel with excipients disclosed herein to obtain the desired physical or (inert ingredient) compositional characteristics of the tablet, caplet or capsules without undue experimentation. In order to improve the flow properties, it may be desirable to pass these blends through a roller compactor or comparable equipment to produce a more flowable material.

Because of the stability properties of prasugrel (susceptibility to hydrolysis and oxidation), certain excipients—most notably povidone and crospovidone (usually containing trace peroxides) and manufacturing processes (e.g. wet granulation) would not be recommended. Barrier bottle packaging performed under positive liquid nitrogen pressures, optionally including an effective desiccant is an advantageous and attractive solution to the problem.

Barrier bottle packages with optional desiccants and optional oxygen scavengers are a convenient presentation for patients and health care providers and these packages can be prepared from a number of materials. Most commonly, bottles are prepared from multi-laminates to produce a finished bottle with the desired handling and permeation characteristics. Conventional HDPE bottles possess excellent moisture resistance, but are quite permeable to the penetration of oxygen. The addition of an intermediate layer of EVOH confers excellent resistance to the permeation of oxygen into the final barrier bottle. Suitable barrier bottles consisting of HDPE/EVOH/HDPE (optionally with other additives or layers) can be obtained from bottle suppliers including Sunoco Products Company (Hartsville, S.C., USA), Alcan Packaging Americas (Coopersburg, Pa., USA), and Takemoto Yohki Co., LTD. (Tokyo, Japan).

After the solid dosage form is filled into the bottles preferably comprising two or more layers of high barrier material (HDPE/EVOH/HDPE) a suitable desiccant may be added to the bottle followed by addition of liquid nitrogen. A cap is then applied to the bottle preferably with an induction seal foil which has low permeability to moisture and oxygen. One of skill in the art is able with minimal experimentation to perform the above procedure(s) including determining the appropriate power settings necessary for the induction sealing while maintaining a positive liquid nitrogen atmosphere.

While a variety of desiccants are available for use with pharmaceutical products (silica gel, activated carbon, clays, molecular sieve, etc.), the ability of these materials to control the relative humidity (RH) in the package headspace can vary greatly. A desiccant material such as molecular sieve has been found to be especially useful because it can maintain the RH of the bottle contents below 10% if adequate quantity is employed and hence would be a preferred desiccant for use in conjunction with prasugrel packaging. One of skill in the art is able to determine with minimal experimentation an adequate quantity of desiccant for the number of tablets in the bottle and the specified shelf life.

Oxygen scavengers are useful as an additional measure to reduce the level of oxygen induced degradation products. Oxygen scavengers packaged in the form of sachets, catridges or canisters may be used for the practice of the present invention. Examples of oxygen scavengers include but is not limited to Ageless™ (Mitsubishi Gas Corporation of Japan) and Pharmakeep™ (Mitsubishi Gas Corporation of Japan). Because of the moisture sensitivity of prasugrel, a preferred oxygen scavenger would have activity at low relative humidities. Of the above oxygen scavengers, Pharmakeep™ is preferred because it has good activity at a wide variety of relative humilities, including the low humidities preferred for packages containing prasugrel. One of skill in the art is able to determine an appropriate amount of oxygen scavenger necessary to effectively maintain oxygen levels below specified limits for the desired length of storage time and for the bottle size and number of tablets or capsules used. For example, U.S. Pat. No. 6,688,468 B2 teaches a method for calculating an approximate amount of oxygen scavenger necessary for a bottle size and for the desired length of storage.

The introduction of positive liquid nitrogen atmosphere (environment) into the barrier bottles containing prasugrel and optional desiccants and/or oxygen scavengers can be accomplished by various means. In one instance, a liquid nitrogen delivery device could be placed on the bottle filling line to introduce liquid nitrogen into the barrier bottles containing solid dosage forms (including optional desiccant and/or oxygen scavengers) just prior to the capping station. By controlling the volume of liquid nitrogen delivered by the liquid nitrogen delivery device, the atmosphere containing oxygen can be displaced from the bottles. This action effectively reduces the oxygen content of the bottles containing the dosage form of prasugrel, optional desiccant and/or oxygen scavenger by filling the headspace with positive nitrogen pressure (from liquid nitrogen). An example of a liquid nitrogen delivery device is the UltraDoser™ 1020 injection system available from VBS Inc, USA. Other liquid nitrogen delivery devices may be available from other manufacturers.

The preferred means for introducing liquid nitrogen into barrier bottles would be to inject a controlled amount of liquid nitrogen into the barrier bottles just prior to the capping station. As the gas heats up and expands, oxygen is effectively reduced by displacement. One of skill in the art is able to set up a filling machine/system such that the entire process of filling with solid dosage form, optional filling with desiccant, optional filling with oxygen scavenger, capping and sealing of the bottle is performed under liquid nitrogen atmosphere to achieve a positive liquid nitrogen pressure.

One of skill in the art is aware that the order of performing certain steps in the manufacturing process may be interchanged except for the step of sealing/capping the bottle under a positive liquid nitrogen atmosphere. For example, the entire process may be performed under a positive liquid nitrogen atmosphere. The steps of adding prasugrel, optional desiccant and optional oxygen scavenger may be performed in any order prior to purging with (adding) liquid nitrogen and sealing the bottle under a positive liquid nitrogen pressure.

While the use of an oxygen scavenger potentially allows for the use of non-barrier bottles (i.e. oxygen permeable containers such as low density polyethylene, high density polyethylene, polypropylene, polystyrene, and polycarbonate containers) and is an embodiment of the present invention, it is preferred that a barrier bottle as described herein be used with a desiccant and/or an oxygen scavenger for effective reductions in oxygen and moisture content.

The stability of bulk drug, tablets, capsules or caplets of prasugrel may be affected by factors including age (length of storage), packaging and storage conditions, such as for example, temperature and relative humidity. The proper packaging and storage conditions ensure an extended shelf life (due to minimized oxygen (<5%) and moisture content) during which the potency of the tablets, caplets or capsules is more likely to be within recommended and/or approved specification limits thereby ensuring the chemical and pharmacodynamic integrity of the tablets, caplets or capsules administered to patients. Specifically, it is now known that stored tablets containing the compound of formula I degrade by both hydrolytic and oxidative pathways. It is also believed that there are crossovers between these degradation pathways wherein intermediates or products of certain steps in one pathway may inter-convert or be kinetically accelerated or hindered by the concentration of product (or intermediate), air or moisture from the environment or the other pathway. While the mechanism of degradation is not fully understood, possible routes of degradation are shown below in Scheme 1.

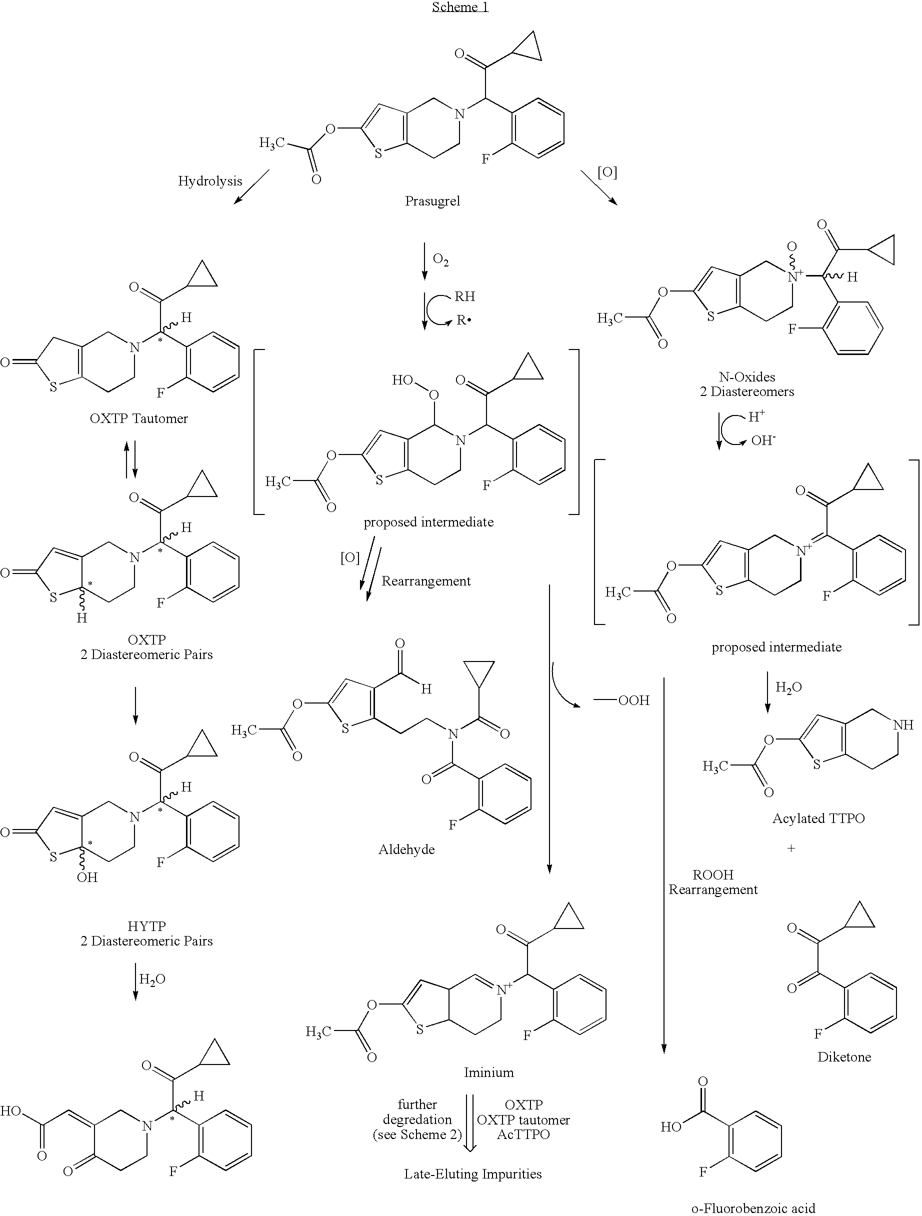

The inventors have measured hydrolytic degradation products classified as OXTP1, and OXTP2, along with the oxidative degradation products classified as Diketone and HYTP respectively. Other products tracked include a collection of less well-defined or and/or unknown late eluting degradation products termed “late eluting impurities” (LEI) as shown in Scheme 1. The inventors have discovered an improved packaging process by tracking the amounts of these degradation products over time under controlled temperature and humidity conditions using packaging methods described herein.

The ideal packaging method would minimize the formation of all degradation products over a longer time period. However, because of the interplay of pathways of degradation and inter-conversion between degradation products and pathways, the next best possibility is to discover a packaging that affords the least change in potency over time. In other words, a preferred objective is to discover a packaging that affords a composite reduction over time in most if not all of the degradation products thereby satisfying a hitherto unmet need. The inventors have achieved the objective of a general reduction in impurity profiles allowing for improved stability and longer shelf life. An additional benefit of the invention is the reduced formation of LEI's which are less well-defined or unknown, uncharacterized and for which specified limits have not been set.

The inventors compared the effect of packaging materials and methods on the stability of a drug product containing prasugrel. The materials and methods compared include (1) liquid nitrogen-inerted 50 mL HDPE bottles containing 25 tablets and a combination desiccant packet containing 0.6 g silica gel and 0.4 g carbon; (2) liquid nitrogen-inerted 75 mL barrier bottles containing 30 tablets and 2 g of molecular sieve desiccant supplied as canisters and inerted with liquid nitrogen. A general packaging description and results for each described package configuration are described below.

Place tablet containing 10 mg of prasugrel in two separate bottle configurations, as described above. The bottle presentations are then placed into controlled environment chambers having the following conditions: 25° C. at 60% relative humidity, 30° C. at 65% relative humidity, and 40° C. at 75% relative humidity for a period of six months. The samples are submitted for chemical analysis to assess changes in potency, total related substances (TRS), OXTP1, OXTP2, Diketone, HYTP and LEI. Data from theses two bottle configurations are presented below in Table 2.

TABLE 2
Bottle Study (HDPE vs. Barrier Bottle)

| Bottle | Packet | | Storage | | Related Substances | | | | | |
|---|---|---|---|---|---|---|---|---|---|---|
| Size/count | Dessicant | | Condition | Potency | TRS-ICH | OXTP1 | OXTP2 | Diketone | HYTP | LEI |
| 1 | 2 | g molecular sieve | 25/60 | 99.1 | 0.12 | 0.09 | 0.12 | 0.03 | 0.04 | 0.17^{a} |
| 2 | 0.6 | g silica/0.4 g carbon | 25/60 | 98.7 | 0.36 | 0.15 | 0.20 | 0.09 | 0.10 | 0.5 |
| 1 | 2 | g molecular sieve | 30/65 | 99.0 | 0.13 | 0.09 | 0.13 | 0.04 | 0.05 | 0.2^{a} |
| 2 | 0.6 | g silica/0.4 g carbon | 30/65 | 99.2 | 0.53 | 0.18 | 0.22 | 0.10 | 0.13 | 0.9 |
| 1 | 2 | g molecular sieve | 40/75 | 98.1 | 0.33 | 0.15 | 0.18 | 0.07 | 0.10 | 0.34^{a} |
| 2 | 0.6 | g silica/0.4 g carbon | 40/75 | 97.0 | 1.11 | 0.31 | 0.34 | 0.12 | 0.35 | 2.7 |

Bottle Size/count: 1 - 75 mL (A)/30- Liquid Nitrogen Interted, (A)- Alcan Barrier (HDPE/EVOH/HDPE) 2 - 50 mL wHDPE/25
TRS-ICH- Only impurities >0.10% are included in this value
^{a}New LEI method was employed. Typically results in levels that are 25-50% lower than when assayed by previous method.

Table 2 shows that the potency of the tablets stored in liquid nitrogen-inerted barrier bottles (A) with molecular sieve desiccants (bottle size/count 1) is generally higher than for tablets stored in the HDPE bottle packaged under normal atmospheric conditions (bottle size/count 2). This is true for most of the storage conditions (25° C., 60% RH, 30° C., 65% RH and 40° C., 75% RH) at the six month time point.

Also, the formation of the related substances is reduced by packaging tablets in barrier bottles that are inerted with liquid nitrogen and containing the molecular sieve desiccant relative to the more conventional packaging components (standard HDPE bottles with silica gel and carbon). This is true for each of the storage conditions (25° C., 60% RH, 30° C., 65% RH and 40° C., 75% RH).

As analytical methods continue to be refined, the impact of packaging on these LEI's is continuing to be elucidated. In general, the improved packaging with barrier bottles containing aggressive desiccants and inerted with liquid nitrogen appears to have a positive impact and reduce the formation of these LEI.

Empty bottles are filled and capped with gaseous nitrogen inerting using three different barrier bottles and a standard HDPE bottle. At representative intervals, the oxygen content of these empty bottles is measured to determine the impact of storage time and conditions on the oxygen concentration in the headspace for each bottle type. Representative results are presented below in Table 3:

TABLE 3
Percent Oxygen Content in Headspace at Indicated Time point
(for Barrier Bottles Inerted and Stored at 40 C.,
75% RH compared to HDPE Bottles Stored at RT)

| | 130 mL Barrier | 100 mL Barrier | 90 mL Barrier | 90 mL |
|---|---|---|---|---|
| | Bottle (S) | Bottle (O) | Bottle (A) | HDPE- |
| Time (Days) | Inerted | Inerted | Inerted | Inerted |
| 0 | 3.67 | 0.68 | 1.62 | 0.674 |
| 7 | | | | 1.4 |
| 10 | 2.2 | | | |
| 14 | | | | 3.26 |
| 22 | | | 2.58 | |
| 30 | | 3.23 | | 4.76 |
| 40 | 3.41 | | | |
| 46 | | | 3.21 | |
| 60 | | 1.84 | | |
| 63 | | | | 8.11 |
| 70 | 2.53 | | | |
| 92 | | | 1.09 | |
| 100 | | 1.68 | | |

Barrier bottle sources: (A) - Alcan Packaging Americas (Coopersburg, PA, USA); O - Owens-Illinois Corporation USA; and (S) - Sunoco Products Company (Hartsville, SC, USA).

The data shows the inability of the HDPE bottle to maintain a low oxygen environment over time thereby discouraging use of this bottle type with inert atmospheres. The study demonstrates that each of three different barrier bottles is able to preserve a low oxygen environment (less than 5%) during subsequent storage in the environmental chambers.

In order to demonstrate the ability of barrier bottles to maintain low oxygen environments in the headspace of packages during storage at various stability conditions, representative packages are sampled at various time points after removal from the stability chambers under various conditions including 25° C., 60% RH, 30° C., 65% RH and 40° C., 75% RH. The results of are presented in the following Table 4.

TABLE 4
Oxygen Content of Barrier Bottles upon
Storage in Environmental Chambers

| Bottle | | | | Storage | Oxygen |
|---|---|---|---|---|---|
| Size/count | Packet Dessicant | Age | | Condition | Content |
| 1 | 2 g molecular sieve | Initial | | na | 3.34% |
| 1 | 2 g molecular sieve | 12.5 | mos | 25/60 | 3.43% |
| 1 | 2 g molecular sieve | 2 | mos | 40/75 | 3.71% |
| 1 | 2 g molecular sieve | 3 | mos | 40/75 | 2.72% |
| 2 | 3 g molecular sieve | Initial | | na | 4.50% |
| 2 | 3 g molecular sieve | 12.5 | mos | 25/60 | 3.45% |
| 2 | 3 g molecular sieve | 2 | mos | 40/75 | 3.38% |
| 2 | 3 g molecular sieve | 3 | mos | 40/75 | 3.31% |
| 1 | 2 g molecular sieve | Initial | | na | 2.12% |
| 1 | 2 g molecular sieve | 12 | mos | 25/60 | 2.96% |
| 1 | 2 g molecular sieve | 2 | mos | 40/75 | 2.41% |
| 1 | 2 g molecular sieve | 4 | mos | 40/75 | 1.89% |
| 2 | 3 g molecular sieve | Initial | | na | 3.30% |
| 2 | 3 g molecular sieve | 12 | mos | 25/60 | 2.76% |
| 2 | 3 g molecular sieve | 2 | mos | 40/75 | 2.52% |
| 2 | 3 g molecular sieve | 4 | mos | 40/75 | 2.57% |

Bottle Size/Count: 1-10 mg- 90 mL Barrier Bottle (A)/30 - Inerted with Liquid nitrogen, 2-10 mg- 90 mL Barrier Bottle (A)/90- Interted with Liquid nitrogen (A) - Alcan Corporation, USA

The above data indicates that suitable barrier bottles inerted using liquid nitrogen are able to maintain low oxygen environments after extended storage at representative ICH ambient and accelerated storage conditions. The oxygen levels in the bottle headspaces appear to be maintained at approximately the initial values, demonstrating minimal oxygen permeation through these intact barrier bottles packaged under a positive liquid nitrogen pressure.

The preferred method of using the invention involves preparing a pharmaceutical package comprising tablets, caplets or capsules of prasugrel as active ingredient manufactured under a positive liquid nitrogen pressure in barrier bottles. The barrier bottles can conveniently be filled with various numbers of tablets and optionally a desiccant to lower the RH of the package headspace and would be dispensed to the patient maintaining package integrity until the product is opened and administered. Typically, the tablet, caplet or capsule may contain from 1 to 20 mg of prasugrel base equivalent. Preferably, the tablet, caplet or capsule may contain from 5 to 15 mg of prasugrel base equivalent. More preferably, the tablet, caplet or capsule contains 5 mg, 7.5 mg, or 10 mg of prasugrel base equivalent.

The following procedures/examples are illustrative of making the tablet, caplet or capsule useful for the practice of the invention and are not intended to limit the scope of the invention in any way. It is understood that the tablets, caplets or capsules so made are then packaged under a positive liquid nitrogen pressure in barrier bottles optionally containing a desiccant and/or oxygen scavenger. Methods of preparing the compound of formula I are known to one of skill in the art having been described in the literature including U.S. Pat. No. 6,693,115 B2.

### EXAMPLES

The following examples are intended to further illustrate the present invention and are not intended to limit the scope of this invention.

### Example 1

Prasugrel HCl (10.98 mg equivalent to 10 mg base), mannitol, hydroxypropyl methylcellulose, croscarmellose sodium, microcrystalline cellulose and magnesium stearate are blended and then roller compacted to produce a granulation. To the resulting granulation, additional croscarmellose sodium, microcrystalline cellulose and magnesium stearate are added and the material is blended and compressed to form tablets weighing from about 175 mg to about 250 mg. An Opadry II® beige film coating mixture is added to water and then sprayed onto these tablets in a side vented coating pan.

The tablets are then packaged in a barrier bottle with a molecular sieve desiccant and then inerted with a liquefied gas such as liquid nitrogen, capped and then sealed using procedures known to one of skill in the art. The following table presents some representative parameters that have been successfully used to inert bottles with liquid nitrogen.

TABLE 5
Inerting with Liquid Nitrogen- prasugrel 10 mg Tablets

| | Bottle Size | Molecular | Liquid | | Distance | | Distance |
|---|---|---|---|---|---|---|---|
| Tablets/ | (Overflow | Sieve | Nitrogen | Dosing | From Nozzle | | from Nozzle |
| Bottle | Capacity) | Quantity | Nozzle¹ | Duration¹ | to Cap | Line Speed | to Capping |
| 4 | 90 mL | 2 g | HFD | 55-65 millisec | ~½ inch | ~15.8 ft/min | ~3 ft |
| 30 | 90 mL | 2 g | HFD | 70-92 millisec | ~½ inch | ~37 ft/min | ~3 ft |
| 90 | 90 mL | 3 g | HFD | 90-110 | ~½ inch | ~54.6 ft/min | ~3 ft |

¹The liquid nitrogen dosing was controlled with a VBS Ultradoser ™ Liquid Nitrogen Injection System (Cryotech VBS International).
HFD—High Flow Divergent

Using the parameters, described in table 5, a total of nine packaging runs were prepared and the following oxygen concentrations were achieved for the various presentations (three lots of each count) as shown below in Table 6:
Table 6-Inerting with Liquid Nitrogen-Oxygen Concentrations Achieved

Prasugrel Tablets Equivalent to 10 mg Base
Packaging Components

| Tablets/Bottle | Molecular Sieve Quantity | Oxygen Concentration |
|---|---|---|
| 4 tablets | 2 g | <4% |
| 30 tablets | 2 g | <3% |
| 90 tablets | 3 g | <4% |

The tablet(s), caplet(s), or capsule(s) are then placed in boxes for storage and/or shipping.

### Example 2

Prasugrel HCl (5.49 mg equivalent to 5 mg base), mannitol, hydroxypropyl methylcellulose, croscarmellose sodium, microcrystalline cellulose and magnesium stearate are blended and then roller compacted to produce a granulation(s). To the resulting granulation(s), additional croscarmellose sodium, microcrystalline cellulose and magnesium stearate are added and the material is blended and compressed to form tablets weighing about 125 mg to about 250 mg. An Opadry II beige film coating mixture is added to water and then sprayed onto these tablets in a side vented coating pan.

The tablets are then packaged in a barrier bottle with a molecular sieve desiccant and then inerted with a liquefied gas such as liquid nitrogen, capped and then sealed using procedures known to one of skill in the art. The following table presents some representative parameters that have been successfully used to inert bottles with liquid nitrogen.

TABLE 7
Inerting with Liquid Nitrogen- Prasugrel 5 mg Tablets

| | Bottle Size | Molecular | Liquid | | Distance | | Distance |
|---|---|---|---|---|---|---|---|
| Tablets/ | (Overflow | Sieve | Nitrogen | Dosing | From Nozzle | | from Nozzle |
| Bottle | Capacity) | Quantity | Nozzle¹ | Duration¹ | to Cap | Line Speed | to Capping |
| 4 | 90 mL | 2 g | HFD | 38-44 millisec | ~½ inch | ~37 ft/min | ~7 ft |
| 30 | 90 mL | 2 g | HFD | 55-70 millisec | ~½ inch | ~37 ft/min | ~3 ft |
| 90 | 90 mL | 3 g | HFD | 62-75 millisec | ~½ inch | ~37 ft/min | ~2 ft |

¹The liquid nitrogen dosing was controlled with a VBS Ultradoser ™ Liquid Nitrogen Injection System (Cryotech VBS International).
HFD—High Flow Divergent

Using the parameters, described in table 7, a total of nine packaging runs were prepared and the following oxygen concentrations were achieved for the various presentations (three lots of each count):

TABLE 8
Inerting with Liquid Nitrogen- Oxygen Concentrations Achieved
Prasugrel HCl Tablets Equivalent to 5 mg Base
Packaging Components

| Tablets/Bottle | Molecular Sieve Quantity | Oxygen Concentration |
|---|---|---|
| 4 tablets | 2 g | <5% |
| 30 tablets | 2 g | <2.5% |
| 90 tablets | 3 g | <4.5% |

The tablet(s), caplet(s), or capsule(s) are then placed in boxes for storage and/or shipping.

### Example 3

Prasugrel HCl (8.24 mg equivalent to 7.5 mg base), mannitol, hydroxypropyl methylcellulose, croscarmellose sodium, microcrystalline cellulose and magnesium stearate are blended and then roller compacted to produce a granulation(s). To the resulting granulation(s), additional croscarmellose sodium, microcrystalline cellulose and magnesium stearate are added and the material is blended and compressed to form tablets weighing from about 125 mg to about 250 mg. An Opadry II beige film coating mixture is added to water and then sprayed onto these tablets in a side vented coating pan.

The tablets are then packaged in a barrier bottle with a molecular sieve desiccant and then inerted with a liquefied gas such as liquid nitrogen, capped and then sealed using procedures known to one of skill in the art. See tables 4 and 5 for examples of the settings that might be employed to inert these barrier bottles with liquid nitrogen. The tablet(s), caplet(s), or capsule(s) are then placed in boxes for storage and/or shipping.

### Example 4

Prasugrel HCl (16.47 mg equivalent to 15 mg base), mannitol, hydroxypropyl methylcellulose, croscarmellose sodium, microcrystalline cellulose and magnesium stearate are blended and then roller compacted to produce granulation(s). To the resulting granulation(s), additional croscarmellose sodium, microcrystalline cellulose and magnesium stearate are added and the material is blended and compressed to form tablets weighing from about 125 mg to about 250 mg. An Opadry II beige film coating mixture is added to water and then sprayed onto these tablets in a side vented coating pan.

The tablets are then packaged in a barrier bottle with a molecular sieve desiccant and then inerted with a liquefied gas such as liquid nitrogen, capped and then sealed using procedures known to one of skill in the art. See tables 4 and 5 for examples of the settings that might be employed to inert these barrier bottles with liquid nitrogen. The tablet(s), caplet(s), or capsule(s) are then placed in boxes for storage and/or shipping.

### Example 5

Prasugrel HCl (32.94 mg equivalent to 30 mg base), mannitol, hydroxypropyl methylcellulose, croscarmellose sodium, microcrystalline cellulose and magnesium stearate are blended and then roller compacted to produce a granulation(s). To the resulting granulation(s), additional croscarmellose sodium, microcrystalline cellulose and magnesium stearate are added and the material is blended and compressed to form tablets weighing from about 125 mg to about 250 mg. An Opadry II beige film coating mixture is added to water and then sprayed onto these tablets in a side vented coating pan.

The tablets are then packaged in a barrier bottle with a molecular sieve desiccant and then inerted with a liquefied gas such as liquid nitrogen, capped and then sealed using procedures known to one of skill in the art. Examples of the settings that might be employed to inert these barrier bottles with liquid nitrogen are found in Tables 4 and 5. The tablet(s), caplet(s), or capsule(s) are then placed in boxes for storage and/or shipping.

### Example 6

Prasugrel HCl (65.88 mg equivalent to 60 mg base), mannitol, hydroxypropyl methylcellulose, croscarmellose sodium, microcrystalline cellulose and magnesium stearate are blended and then roller compacted to produce a granulation(s). To the resulting granulation(s), additional croscarmellose sodium, microcrystalline cellulose and magnesium stearate are added and the material is blended and compressed to form tablets weighing from about 250 mg to about 500 mg. An Opadry II beige film coating mixture is added to water and then sprayed onto these tablets in a side vented coating pan.

The tablets are then packaged in a barrier bottle with a molecular sieve desiccant and then inerted with a liquefied gas such as liquid nitrogen, capped and then sealed using procedures known to one of skill in the art. Tables 4 and 5 provide examples of the settings that might be employed to inert these barrier bottles with liquid nitrogen. The tablet(s), caplet(s), or capsule(s) are then placed in boxes for storage and/or shipping.

1. An article of manufacture comprising tablets, caplets, capsules or other solid form of prasugrel or an air and/or moisture sensitive pharmaceutical agent packaged in an air and/or moisture impervious container under a positive liquid gas pressure.

2. An article of manufacture according to claim 1 comprising tablets, caplets, capsules or other solid form of prasugrel packaged in an air and/or moisture impervious container under a positive liquid gas pressure.

3. An article of manufacture according to claim 2 wherein the liquid gas is liquid nitrogen.

4. An article of manufacture according to claim 2 further comprising from about 5 mg to about 10 mg base equivalent of prasugrel per tablet, caplet, capsule, or other solid form of prasugrel.

5. An article of manufacture according to claim 2 wherein the oxygen content of the headspace in the air and/or moisture impervious container containing prasugrel tablets caplets, capsule or other solid forms is less than 5%.

6. An article of manufacture according to claim 2 wherein the oxygen content of the headspace in the air and/or moisture impervious container containing prasugrel tablets caplets, capsule or other solid forms is less than 4%.

7. An article of manufacture according to claim 2 comprising 5 mg, 7.5 mg, or 10 mg base equivalent tablets, caplets, capsules, or other solid form of prasugrel packaged in an air and/or moisture impervious container under a positive liquid nitrogen pressure.

8. An article of manufacture according to claim 2 wherein the air and/or moisture impervious container is a bottle.

9. An article of manufacture according to claim 8 wherein the bottle is a multilayer bottle comprising HDPE/EVOH/HDPE.

10. An article of manufacture according to claim 2 further comprising a desiccant packaged within the air and/or moisture impervious container.

11. An article of manufacture according to claim 10 wherein the desiccant is a molecular sieve.

12. An article of manufacture according to claim 2 further comprising an oxygen scavenger packaged within the air and/or moisture impervious container.

13. A process for the manufacture of tablets, caplets, capsules or other solid form of prasugrel containing from about 5 mg to about 10 mg of prasugrel comprising the steps of:
a. placing tablets, caplets, capsules or other solid form of prasugrel in an air and/or moisture impervious bottle;
b. optionally adding a desiccant;
c. optionally adding an oxygen scavenger;
d. adding liquid nitrogen, and
e. sealing the air and/or moisture impervious bottle under a positive liquid nitrogen pressure.

14. A process according to claim 13 wherein the container is a bottle.

15. A process according to claim 13 wherein the oxygen content of the headspace in the air and/or moisture impervious bottle containing prasugrel produced by the process is less than 5%.

16. A process according to claim 13 wherein the oxygen content of the headspace in the air and/or moisture impervious bottle containing prasugrel produced by the process is less than 4%.

17. A process for improving the stability of a tablet, caplet, capsule or other solid form of prasugrel comprising the steps of:
a. placing a tablet, caplet, capsule or other solid form of prasugrel in an air and/or moisture impervious bottle;
b. optionally adding a desiccant;
c. optionally adding an oxygen scavenger,
d. adding liquid nitrogen; and
e. sealing the air and/or moisture impervious bottle under a positive liquid nitrogen pressure.

18. A process according to claim 17 wherein the container is a bottle.

19. A process according to claim 17 wherein the oxygen content of the headspace in the air and/or moisture impervious bottle containing prasugrel is less than 5%.

20. A process according to claim 17 wherein the oxygen content of the headspace in the air and/or moisture impervious bottle containing prasugrel is less than 4%.

21. A process for improving the shelf life of tablets, caplets, capsules or other solid form of prasugrel comprising the steps of:
a. placing a tablet, caplet, capsule or other solid form of prasugrel in an air and/or moisture impervious container;
b. optionally adding a desiccant;
c. optionally adding an oxygen scavenger,
d. adding liquid nitrogen; and
e. sealing the air and/or moisture impervious container under a positive liquid nitrogen pressure.

22. A process according to claim 21 wherein the container is a bottle.

23. A process according to claim 21 wherein the oxygen content of the headspace in the air and/or moisture impervious bottle containing prasugrel is less than 5%.

24. A process according to claim 21 wherein the oxygen content of the headspace in the air and/or moisture impervious bottle containing prasugrel is less than 4%.

25. A pharmaceutical kit comprising a tablet, caplet, capsule or other solid form of prasugrel packaged in an air and/or moisture impervious bottle under a positive liquid nitrogen pressure.
